(19) United States
(12) Patent Application Publication
(10) Pub. No.: US 2025/0377289 A1
(43) Pub. Date: Dec. 11, 2025

(54) METHODS AND SYSTEMS FOR OPTICAL ANALYSIS

(71) Applicant: Elio Inc., San Mateo, CA (US)

(72) Inventor: Nadav GROSSINGER, Karmei Yosef (IL)

(73) Assignee: Elio Inc., San Mateo, CA (US)

(21) Appl. No.: 18/875,287

(22) PCT Filed: Jun. 16, 2023
(86) PCT No.: PCT/IB2023/056258

§ 371 Date: Dec. 16, 2024

Related U.S. Application Data
(60) Provisional application No. 63/352,759, filed on Jun. 16, 2022.
(60) Provisional application No. 63/386,442, filed on Dec. 7, 2022.

(51) Int. Cl. G01N 21/01; G01N 33/53

(52) U.S. Cl. CPC G01N 21/01; G01N 33/5308; G01N 2201/0636; G01N 2201/1296

(57) ABSTRACT

In an aspect, the present disclosure can provide a method of detecting a biomolecule. A sample can be provided comprising the biomolecule. Electromagnetic radiation can be directed to the sample, thereby interacting the electromagnetic radiation with the biomolecule. The electromagnetic radiation can be propagated through the diffractive decoder, thereby generating an optical output. A presence or absence of the biomolecule in the sample can be detected based at least in part on the optical output.

### RELATED APPLICATIONS

This application claims the benefit of priority of U.S. Provisional Patent Application No. 63/352,759 filed on 16 Jun. 2022 and U.S. Provisional Patent Application No. 63/386,442 filed on 7 Dec. 2022, the contents of which are incorporated herein by reference in their entirety.

### BACKGROUND

Diagnostic testing methods and devices have become an important part of modern medical care. Analysis of non-labeled biological samples can provide insight into the properties of the biological sample.

### SUMMARY

In an aspect, the present disclosure provides a method of detecting a biomolecule, comprising: (a) providing a sample comprising the biomolecule; (b) directing electromagnetic radiation to the sample, thereby interacting the electromagnetic radiation with the biomolecule; (c) propagating the electromagnetic radiation through a diffractive decoder, thereby generating an optical output; and (d) detecting a presence or absence of the biomolecule in the sample based at least in part on the optical output.

In some embodiments, the diffractive decoder comprises a waveguide. In some embodiments, the waveguide is a one dimensional or two dimensional waveguide. In some embodiments, the diffractive decoder comprises a plurality of layers. In some embodiments, the diffractive decoder comprises a plurality of nano-printed features. In some embodiments, the biomolecule is selected from the group consisting of a nucleic acid molecule, a protein, and an antigen. In some embodiments, the sample comprises one or more of blood, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, other excretions or body tissues, or any combination thereof. In some embodiments, the detecting comprises use of an optical detector. In some embodiments, the optical detector comprises a photomultiplier tube (PMT), photodiode, avalanche diode, single-photon avalanche diode, single-photon avalanche diode array, phototransistor, reverse-biased light emitting diode (LED), charge coupled device (CCDs), or complementary metal oxide semiconductor (CMOS) camera. In some embodiments, the optical detector converts the optical output to an electrical output. In some embodiments, the electromagnetic radiation comprises light. In some embodiments, the light comprises multiple wavelengths of light. In some embodiments, the light is structured light. In some embodiments, the method further comprises determining a quantity or concentration of the biomolecule. In some embodiments, the electromagnetic radiation is transmitted through the sample. In some embodiments, the electromagnetic radiation is reflected off the sample. In some embodiments, (a)-(d) occurs in less than about 1 minute. In some embodiments, (a)-(d) occurs in less than about 1 second. In some embodiments, (a)-(d) occurs in less than about 0.1 seconds.

In another aspect, the present disclosure provides a diffractive decoder, comprising: a waveguide comprising a plurality of physical features formed therein, wherein a physical arrangement of the plurality of physical features collectively defines a trained neural network, wherein the trained neural network is configured to propagate an input optical signal through the waveguide to generate an output optical signal.

In some embodiments, the diffractive decoder further comprises a detector. In some embodiments, the diffractive decoder further comprises a plurality of detectors. In some embodiments, the plurality of detectors are positioned at a plurality of locations in or adjacent to the waveguide. In some embodiments, the diffractive decoder further comprises a plurality of holes in the waveguide, wherein the plurality of holes are in optical communication with the detector. In some embodiments, the detector comprises a photomultiplier tube (PMT), photodiode, avalanche diode, single-photon avalanche diode, single-photon avalanche diode array, phototransistor, reverse-biased light emitting diode (LED), charge coupled device (CCDs), or complementary metal oxide semiconductor (CMOS) camera. In some embodiments, the waveguide is configured to propagate the input optical signal in one dimension. In some embodiments, the waveguide is configured to propagate the input optical signal in two dimensions. In some embodiments, the waveguide comprises gold, silver, a dielectric material, or any combination thereof. In some embodiments, the waveguide is a cylinder. In some embodiments, the waveguide is a prism. In some embodiments, the waveguide is planar. In some embodiments, the diffractive decoder further comprises a mirror positioned within the waveguide. In some embodiments, the mirror is configured to reflect the input optical signal back through the waveguide. In some embodiments, the output optical signal is configured for use as illumination light. In some embodiments, the output optical signal is configured to be detected by a detector. In some embodiments, the physical features are not adjustable with respect to one another. In some embodiments, the diffractive decoder further comprises a sample area in optical communication with the waveguide. In some embodiments, the sample area is in transmissive communication with the waveguide. In some embodiments, the sample area is in reflective communication with the waveguide.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE SEVERAL VIEWS OF THE DRAWING(S)

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a computer system that is programmed or otherwise configured to implement methods provided herein.

FIG. 2 depicts an example method of detecting a biomolecule.

FIGS. 3A-3D show examples of various designs for diffractive decoders.

FIGS. 4A-4C show examples of different views of a diffractive decoder.

FIG. 5 shows an example of an iterative process for designing diffractive decoders.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

### Computer Systems

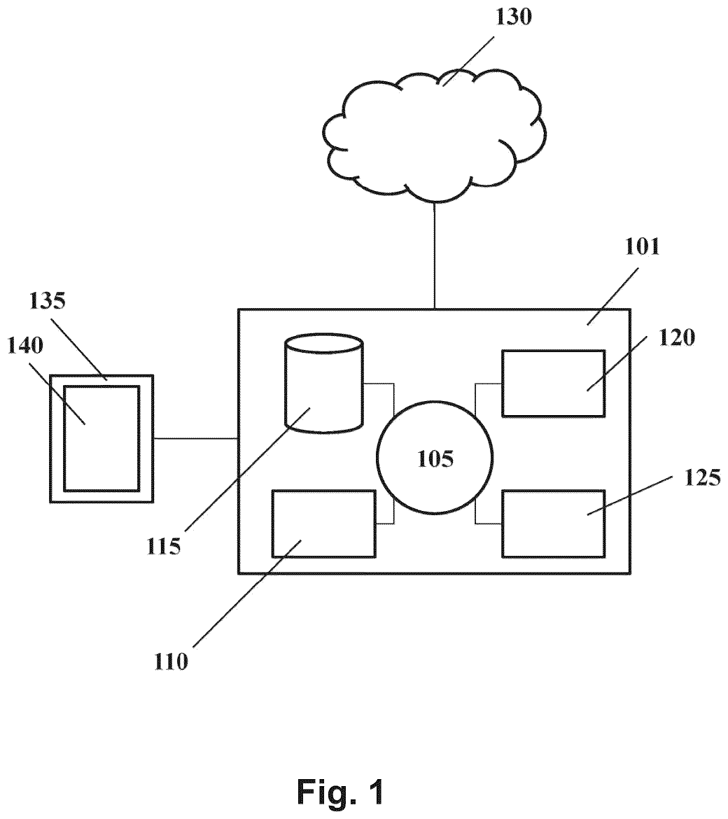

The present disclosure provides computer systems that are programmed to implement methods of the disclosure. FIG. 1 shows a computer system 101 that is programmed or otherwise configured to implement the methods described elsewhere herein. The computer system 101 can regulate various aspects of the present disclosure, such as, for example, an optical system operatively coupled to an optical computer. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, an interface for receiving an output of an analysis. Examples of UI's include, without limitation, a graphical user interface (GUI) and web-based user interface.

### Example Method for Detecting a Biomolecule

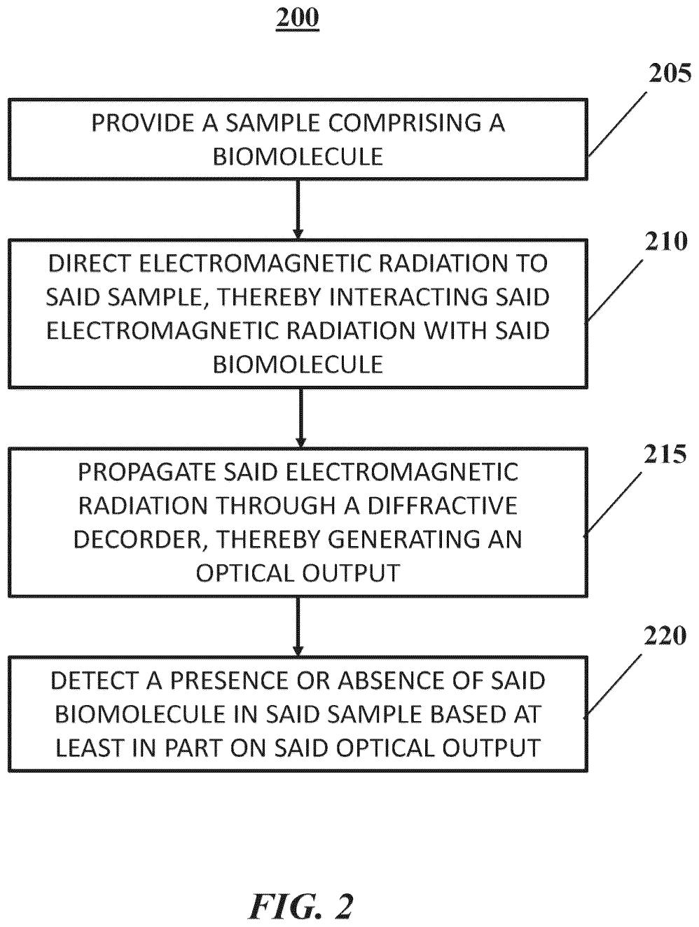

FIG. 2 depicts an example method 200 of detecting a biomolecule. At a high level, the method may include (a) providing a sample comprising the biomolecule (block 205); (b) directing electromagnetic radiation to the sample, thereby interacting the electromagnetic radiation with the biomolecule (block 210); (c) propagating the electromagnetic radiation through a diffractive decoder, thereby generating an optical output (block 215); and (d) detecting a presence or absence of the biomolecule in the sample based at least in part on the optical output (block 220). Some aspects of the method 200 may be implemented using a computer system that may be the same as or similar to the computer system 101 of FIG. 1. In some cases, the method may be for other applications from detecting a biomolecule. For example, a similar method can be for detecting structural features and/or changes thereof within a cell of a sample. In another example, a spatial and/or volumetric arrangement of cells in a sample can be probed.

In some cases, the method 200 may begin with providing the sample comprising the biomolecule at block 205. The sample may be any type of biological sample, such as human, plant or animal sample. The sample may be tissue of a living (or previously-living) organism or excretion from a living (or previously-living) organism. Referring to the case where the sample originates from a human or animal, the sample may include one or more of: blood, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, etc. The sample may be of various different sizes or quantities and may have been collected at one time or various different times.

The sample may include one or more different types of biomolecules. The biomolecules may be organic molecules that may be assist in or be important to the survival of living cells or living organisms. For example, the biomolecule may be a nucleic acid molecule, a protein, an antigen, a lipid, a carbohydrate, etc. The biomolecules may be of various amounts or concentrations in the sample.

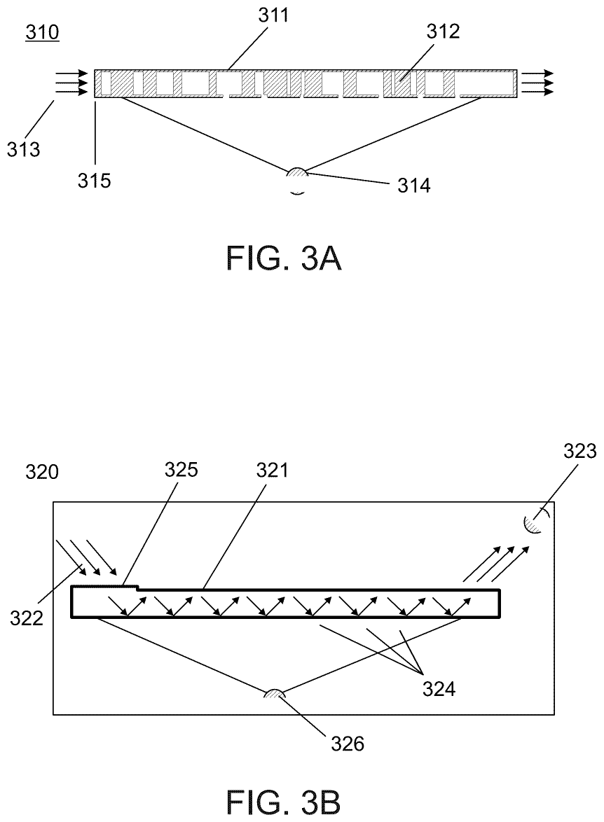

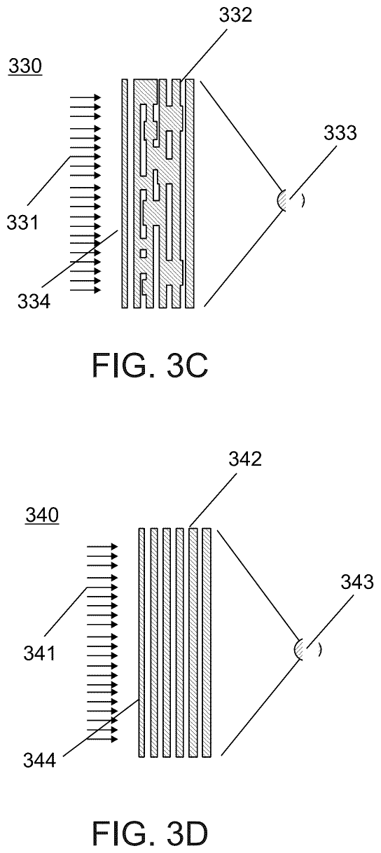

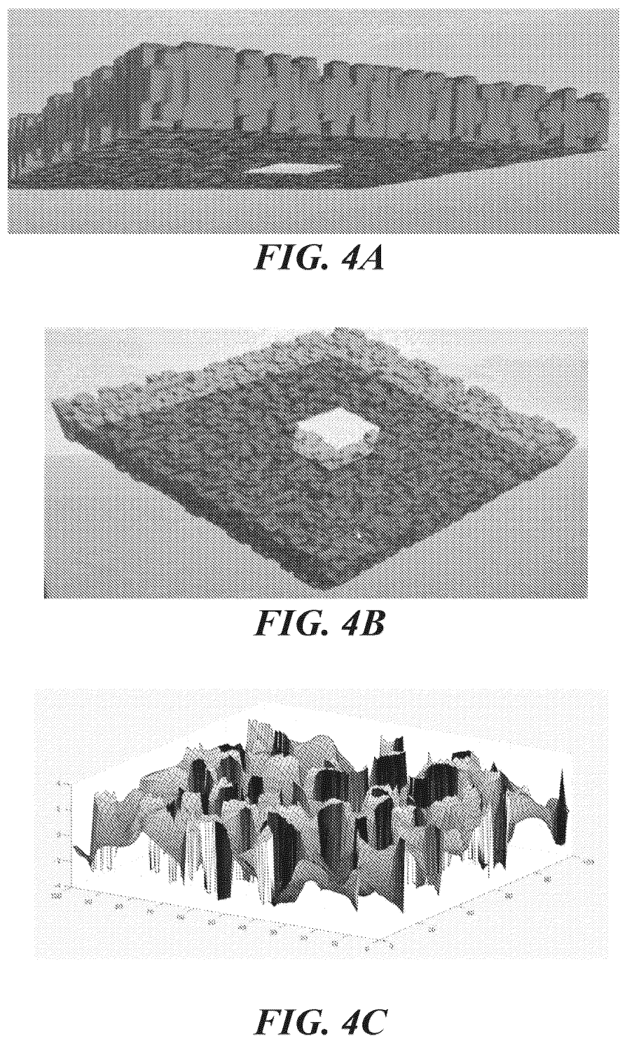

The sample may be provided to a system (e.g., the systems of FIGS. 3-4) that includes one or more of an electromagnetic radiation source, a diffractive decoder, or an optical detector. The sample may be provided to the system by a human (e.g., a technician) or by an automated device (e.g., a robotic arm, a conveyor system, etc.). The sample may be provided to the system as a single sample or as part of a batch of other samples. The sample may be provided to the system for use in medical, scientific, or research contexts.

In some cases, the method 200 may include directing the electromagnetic radiation to the sample, thereby interacting the electromagnetic radiation with the biomolecule at block 210. The electromagnetic radiation may comprise light of a single wavelength. Alternatively, the electromagnetic radiation may comprise light of a plurality of wavelengths. The electromagnetic radiation may comprise light that is structured light. In some cases, the electromagnetic radiation may comprise any form of ionizing or non-ionizing radiation over the electromagnetic spectrum such as radio waves, microwaves, infrared, visible light, ultraviolet, x-rays, or gamma rays. In some cases, the electromagnetic radiation may include one or more types of radiation over the electromagnetic spectrum or one or more wavelengths. The light may comprise polarized light (e.g., linearly polarized light, circularly polarized light, etc.). The polarization of the light may be a property usable in the diffractive decoders described elsewhere herein. For example, the polarization of light can be affected by a diffractive decoder to determine a property of a sample as described elsewhere herein.

In cases where the electromagnetic radiation includes light, the light may be provided by any number of light sources that produce photons from another energy source, such as heat, chemical reactions, or conversion of mass or a different frequency of electromagnetic energy. The light sources may be incandescent, as in emitting light from a hot body as a result of temperature, or luminescent, as in emitting light by a substance not resulting from heat. Examples of incandescent light sources may include one or more of: combustion light sources, lamps, burners, candles, fire, embers, incandescent light bulbs, explosives, nuclear or high energy particles (e.g., nuclear fusion, nuclear fission, free-electron laser), or celestial or atmospheric sources (e.g., the sun).

Examples of luminescent light sources may include one or more of: bioluminescence, cathodoluminescence, chemiluminescence, cryoluminescence, crystalloluminescence, electric discharge (e.g., arc lamp, fluorescent lamp, hollow-cathode lamp, neon and argon lamp, etc.), electrochemiluminescence, electroluminescence (e.g., light-emitting diode, laser), mechanoluminscence, photoluminescence, radioluminescence, or thermoluminescence. In some cases, the electromagnetic radiation is provided by one or more lasers that may be one or more of: chemical laser, dye laser, free-electron laser, gas dynamic laser, gas laser, ion laser, laser diode, laser flashlight, metal-vapor laser, nonlinear optical laser, quantum well laser, quantum dot laser, ruby laser, solid-state laser, or any other suitable type of laser.

In some cases, the electromagnetic radiation (e.g., light) may be directed to the sample by pointing the electromagnetic radiation sources (e.g., one or more lasers) towards the sample. In other cases, the electromagnetic radiation may be directed to the sample using guides such as physical waveguides, opaque objects, mirrors, filters, or other suitable devices or techniques for directing light. In some cases, the electromagnetic radiation is not direct, but rather indirect (e.g., ambient) electromagnetic radiation. When the electromagnetic radiation reaches the sample, and accordingly, the biomolecule, the electromagnetic radiation may interact with the biomolecule. Interaction of the electromagnetic radiation with the biomolecule may include one or more of: diffraction, reflection, emission, absorption, transmission, refraction, interference, etc., with one or both of the biomolecule or the electromagnetic radiation itself. For example, in some cases, the electromagnetic radiation may be transmitted through the sample. In another example, in some cases, the electromagnetic radiation may be reflected off the sample.

In some cases, the method 200 may include propagating the electromagnetic radiation through the diffractive decoder, thereby generating the optical output at block 215. The diffractive decoder may be in one or more of optical communication, transmissive communication, or reflective communication with the diffractive decoder. Propagating the electromagnetic radiation through the diffractive decoder may be done over any number of dimensions. For example, the diffractive decoder may be configured to propagate the electromagnetic radiation in one dimension. In another example, the diffractive decoder may be configured to propagate the electromagnetic radiation in a plurality of dimensions, such as two dimensions, three dimensions, or four dimensions. In some cases, the diffractive decoder may comprise one or more layers (e.g., about two layers, about ten layers, about one hundred layers, about one thousand layers, about one million layers, etc.).

In some cases, the diffractive decoder does not include any layers, thereby reducing challenges associated with inter layer alignment in the diffractive decoder using the non-layer architecture. For example, the diffractive decoder can be configured with a single region of diffractive features not configured to be placed into an optical stack. In this example, the diffractive decoder can be configured with diffractive features on one side of the diffractive decoder, and a mirror can be used to reflect the light back onto the diffractive decoder to provide a plurality of virtual decoder layers. The lack of layers can provide benefits including, but not limited to, improved ease of alignment, increased setup precision, etc. In some cases, preparation or fabrication of the diffractive decoder may include nano-printing the diffractive decoder, and, accordingly, the diffractive decoder may comprise various nano-printed features. In some cases, lithographic generation techniques may be used to prepare or fabricate the diffractive decoder.

The diffractive decoder may be modular. For example, a system comprising the diffractive decoder can be configured to receive a plurality of diffractive decoders in a predetermined location of the system. For example, the system can be configured to accept a diffractive decoder as a cartridge in the system, thereby permitting different diffractive decoders to be used in a same system to interrogate different properties of a sample. In an example, a first diffractive decoder can be slotted into a system to determine a presence of a biological molecule, and a second diffractive decoder can be slotted into the system to determine a cellular structure of the sample. In this example, the sample may be stationary during the changing of the diffractive decoders.

In some cases, the diffractive decoder may comprise a waveguide. The waveguide may be may one or more of: a rectangular waveguide, a circular waveguide, an elliptical waveguide, a single-ridged waveguide, or a double-ridged waveguide. In some cases, the waveguide may be a cylinder. In some cases, the waveguide may be a prism. In some cases, the waveguide may be planar. The waveguide may be comprised of any number of suitable materials including gold, silver, a dielectric material, brass, copper, silver, aluminum, polymer, plastic, carbon fiber, or any combination thereof. The waveguide may be comprised of a plurality of physical features formed therein. The physical features may be not adjustable with respect to one another or are fixed in place. In some cases, the waveguide may include one or more holes. For example, a two-dimensional waveguide may have small holes configured to allow electromagnetic radiation to escape prior to passing through the remainder of the waveguide or the system (e.g., the system of any one of FIGS. 3-4) as a whole. In some cases, the waveguide can comprise a plurality (e.g., two or more) mirrors configured to form the waveguide by reflecting the light between the mirrors. For example, two mirrors can be placed parallel to one another, and the light can be guided by reflection between the two mirrors. In some cases, one or more mirrors of the waveguide can be controllable mirrors. The controllable mirrors may be configured to have one or more properties of the mirrors adjusted (e.g., adjustable position, reflectance, transmission, etc.). Examples of controllable mirrors include, but are not limited to controllable dynamic micromirrors, spatial light modulators, phase modulators, or the like, or any combination thereof. In some cases, a reconfigurable diffractive decoder can be reconfigured remotely from the generation of the diffractive decoder. For example, a diffractive decoder comprising dynamic micromirrors can be produced and supplied to a user and, subsequent to the delivery to the user, reconfigured. For example, a diffractive decoder can be reconfigured to permit retraining of the diffractive decoder after generation of the diffractive decoder.

In some cases, a physical arrangement of the plurality of physical features of the diffractive decoder (e.g., the waveguide) collectively defines a trained machine learning (ML) model, such as a trained neural network. The trained ML model may generally be any system or computational procedure that takes one or more actions to enhance or maximize a chance of successfully achieving a goal, such as a system or analytical and/or statistical procedure that progressively improves computer performance of a task. The ML model may comprise one or more supervised, semi-supervised, or unsupervised machine learning techniques. For example, the ML model may be a trained algorithm that is trained through supervised learning (e.g., various parameters are determined as weights or scaling factors).

The ML model may comprise one or more techniques of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principle component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks. A neural network as described elsewhere herein can comprise at least a portion of the neural network as an optical neural network. The optical neural network may be configured to facilitate a guided interference between portions of an input light wavefront and the sample. For example, the optical neural network can be configured to facilitate interference of the input wavefront subsequent to interaction with a sample in order to determine a property of the sample upon detection of the wavefront after interaction with the optical neural network.

Not only may the diffractive decoder define a trained ML model via the plurality of physical features included therein, but, in some cases, the diffractive decoder itself may be designed using a ML model. The ML model may be used to design one or more characteristics of the diffractive decoder. For example, the ML model may be used to design the materials, size, weight, length, density, dimensions, hardness, shape, or other optical, mechanical, material, chemical, optical, and/or physical properties of the diffractive decoder. In some cases, characteristics (e.g., location, size, number, shape, etc.) of the holes of the diffractive decoder may be designed by a ML model.

The diffractive decoder may be designed according to an iterative method or process. For example, a ML model (which may be the same as or similar to one or more of the ML models described herein) may be used to design a first iteration of the diffractive decoder. The first iteration may be tested or validated (e.g., by human operation, automation, etc.) via any number of techniques. For example, if the first iteration of the diffractive decoder defines a trained ML mode, the first iteration may be tested or validated using one or more holdout or validation data sets to determine if the diffractive decoder satisfies certain specifications, thresholds, or performance guidelines.

If the first iteration does not satisfy the certain specifications, thresholds, or performance guidelines, then a second iteration diffractive decoder may be designed (e.g., by modifying parameters of the first iteration diffractive decoder, modifying parameters of the ML model that designed the first iteration, or by modifying the first iteration diffractive decoder directly). Again, the second iteration diffractive decoder may be tested against the certain specifications, thresholds, or performance guidelines, and the process may continue iteratively. The ML model may design the diffractive decoder to optimize for one or more objectives (e.g., the certain specifications, thresholds, or performance guidelines), such as one or more of detectability of the biomolecule, false-positive rate, false-negative rate, cost, material use, computational energy, computational time, etc. In some cases, the ML model may optimize for the objectives using constrained optimization techniques to respect certain constraints (e.g., size, resource, time, etc.).

As described herein, the trained neural network, defined by the diffractive decoder, may be configured to propagate an input optical signal (e.g., the electromagnetic radiation) through the diffractive decoder to generate the optical output, which may be configured for use as illumination light. In some cases, the optical output may be reflected back through the diffractive decoder via a mirror that is positioned within the diffractive decoder. In some cases, the mirror may be on the top surface of the diffractive decoder so that the electromagnetic radiation can traverse the diffractive decoder.

In some cases, the method 200 may include detecting a presence or absence of the biomolecule in the sample based at least in part on the optical output at block 220. Detecting the presence or absence of the biomolecule in the sample may use one or more detectors. The optical output may be configured to be detected by the detectors. The detectors may include one or more optical detectors. At a high level, the optical detectors may convert the detected optical output to a different type of output. For example, the optical detectors may convert the optical output to an electrical output. The optical detectors may include one or more of a photomultiplier tube (PMT), photodiode, avalanche diode, single-photon avalanche diode, single-photon avalanche diode array, phototransistor, reverse-biased light emitting diode (LED), charge coupled device (CCDs), a time of flight (TOF) sensor, or complementary metal oxide semiconductor (CMOS) camera.

In some cases, the detectors may be positioned at one or more locations in or adjacent to the diffraction decoder (e.g., the waveguide). In some cases, the holes in the diffraction decoder may be in optical communication with the detectors. The holes may enable creation of a “virtual” detector array that addresses one or more of the holes. The detectors, may, in some cases, be parasitic in that the detectors absorb the electromagnetic radiation. Similar to how the ML model may be used to design the diffractive decoder (e.g., design the parameters or characteristics of the diffractive decoder), the ML model may be used to design the layout (e.g., positioning, spacing, sizing, etc.) of the detectors. The ML model may design the layout of the detectors to optimize for one or more objectives, such as one or more of detectability of the biomolecule, false-positive rate, false-negative rate, cost, material use, computational energy, computational time, etc.

In some cases, a single type of biomolecule (e.g., lipids) may be detected by the method 200. In some cases, the method 200 may enable detection of a plurality of types of biomolecules (e.g., capable of detecting lipids and proteins). While in some cases, the method 200 may detect a presence or absence of the biomolecule, in other cases, in other cases, the method 200 may determine how much (e.g., concentration, quantity, mass, number of molecules, etc.) of the biomolecule is present in the sample. As discussed herein, computational time may be an important objective (e.g., the certain specifications, thresholds, or performance guidelines) or consideration. As such, various aspects of the present techniques may be adapted (e.g., using a ML model to design one or more aspects of the present techniques, as described herein) to complete the method 200 in a certain amount of time. In some cases, the method 200 may be executed in less than about one minute. In some cases, the method 200 may be executed in less than about one second. In some cases, the method 200 may be executed in less than about 0.1 seconds.

Methods and systems of the present disclosure can be implemented by way of one or more algorithms. An algorithm can be implemented by way of software upon execution by the central processing unit 105. The algorithm can, for example, interpret an output of an optical computer.

FIGS. 3A-3D show examples of various designs for diffractive decoders 310, 320, 330, and 340. Diffractive decoder 310 may be an example of a one-dimensional diffractive decoder. The one-dimensional diffractive decoder may be configured to accept input light 313. The input light may be light as described elsewhere herein. For example, the input light can be unstructured visible light, structured visible light, or the like. The input light 313 can interact with sample 315. The sample can be as described elsewhere herein. Upon interaction with the sample, the light can propagate through the waveguide 311 comprising physical features 312 (e.g., physical features as described elsewhere herein). For example, the light can interact with the sample, and the sample can impart properties on the light (e.g., phase, etc.), and the light can interact with the physical features of the diffractive decoder. Upon propagation through the physical features, the light can exit the waveguide and be detected by a detector 314. The detector can be as described elsewhere herein. The detector can be positioned to detect the light upon full transmission through the waveguide (e.g., after the light has interacted with the physical features and has traveled the entire length of the waveguide) and/or when the light has partially traversed the waveguide (e.g., after the light has exited the waveguide at a point prior to the end of the waveguide).

FIG. 3B shows an example of a two-dimensional diffractive decoder 320. The two-dimensional diffractive decoder may continue in and/or out of the plane of FIG. 3B (e.g., form a three dimensional object comprising a two-dimensional array of physical features). Incident light 322 can interact with a sample 325, and the light transmitted through the sample can interact with the physical features 321. In some cases, the diffractive decoder can be configured where the incident light 322 can interact with the sample 325 in a transmissive configuration (e.g., the light transmits through the sample prior to interacting with the physical features). In some cases, the diffractive decoder can be configured where the incident light 322 can interact with the sample 325 in a reflective configuration (e.g., the light can reflect off of the sample prior to interacting with the physical features). The light can be configured to interact with the physical features 321 a plurality of times (e.g., by reflecting through the diffractive decoder as indicated by the arrows). In some cases, the light can propagate through the diffractive decoder, interacting multiple times with the physical features, before exiting the diffractive decoder and being detected by a detector 323.

In some cases, the diffractive decoder can comprise features 324, which can be configured to permit at least a portion of the light to escape the diffractive decoder and be detected by detector 326. The features 324 may comprise openings (e.g., slits, holes, etc.), semitransparent features (e.g., dichroic mirrors, beamsplitters, partially reflective mirrors, etc.), or the like, or any combination thereof. For example, the features can comprise partially reflective mirrors placed above holes in the diffractive decoder. In this example, a portion of the light can be removed through the partially reflective mirror and detected, while the rest of the light is further propagated and further interacts with the physical features. In this example, the light collected through the features by detector 326 may provide different information from the light collected by detector 323. The two-dimensional diffractive decoder may provide improvements including high efficiency (e.g., high efficiency of interaction of the light with the physical elements), many layers of interaction (e.g., many interactions between the light and the physical features in a small footprint), easier alignment (e.g., not having to align a plurality of layers of diffractive physical features), and the like.

FIG. 3C shows an example of a nano-printed diffractive decoder 330. The light 331 can interact with a sample 334 before propagating through physical features 332 and being detected by detector 333. The physical features may comprise one or more nano-printed physical features. The nano-printed physical features can comprise features generated using a nano-print apparatus. For example, a nano-imprint system can imprint features onto optically transmissive substrates, which can then be aligned into the array of physical features. The nano-printed diffractive decoder can provide flexible structures (e.g., easy tuning of the structure for different applications) as well as high densities of computation.

FIG. 3D shows an example of a layer based diffractive decoder 340. The light 341 can interact with a sample 344 before propagating through a plurality of layers comprising physical features 342 and subsequently being detected by detector 343. The layer based diffractive decoder can provide simple integration and modularity.

The following examples are illustrative of certain systems and methods described herein and are not intended to be limiting.

### EXAMPLE 1—ARCHITECTURE OF A DIFFRACTIVE DECODER

FIGS. 4A-4C show examples of different views of a diffractive decoder. FIGS. 4A and 4B can show different views of a diffractive decoder. The features of the diffractive decoder can be observed in the figures. The features may be responsible for at least a portion of the differences in the optical properties (e.g., diffractive properties) of the diffractive decoder. For example, the differing height of the features can provide for different effective path lengths within the diffractive decoder, which can, in turn, generate different light paths depending on the incident light. FIG. 4C shows a detail of an example surface of a diffractive decoder. The profile of the diffractive decoder can comprise an array of shapes (e.g., triangles, squares, pentagons, hexagons, other polygons, etc.), gradients (e.g., non-Euclidean shaped gradients), other arbitrary forms, or the like, or any combination thereof. In the example of FIG. 4C, the profile can comprise a plurality of arbitrary height profile gradients.

### EXAMPLE 2—ITERATIVE DESIGN OF DIFFRACTIVE DECODERS

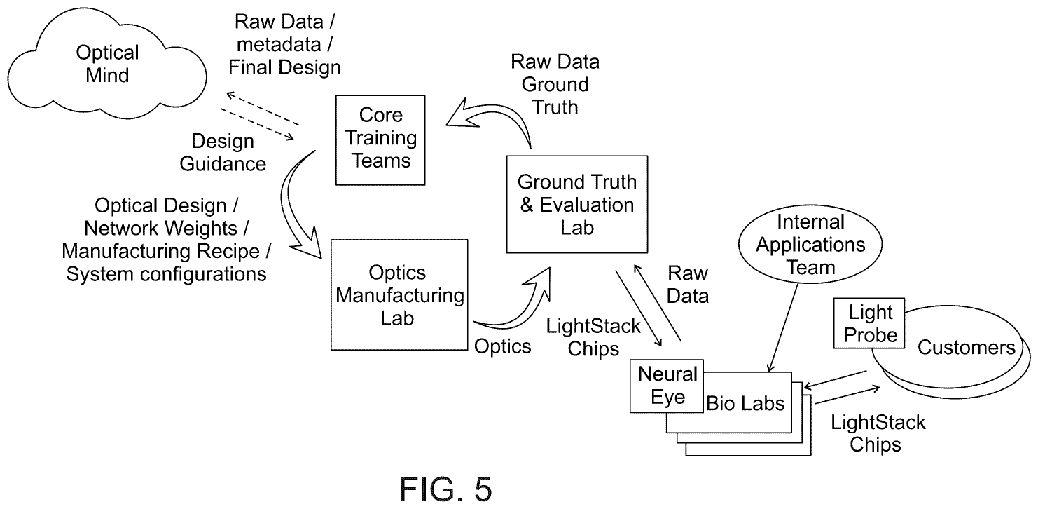

FIG. 5 shows an example of an iterative method or process for designing diffractive decoders. The iterative process can be configured to develop and/or optimize one or more diffractive decoders for one or more predetermined purposes. For example, the iterative process can be used to develop a first diffractive decoder for a first predetermined purpose and a second diffractive decoder for a second predetermined purpose.

The iterative process may comprise determining an application for a diffractive decoder. For example, the process can be used to design a diffractive decoder to detect a particular biomolecule. An initial design for the diffractive decoder can be determined by stochastic generation, user guided generation, algorithm guided generation, or any combination thereof. For example, a random array can be generated as a starting point in the design of the diffractive decoder.

Subsequent to the generation of the initial design, the design can be iterated upon using one or more machine learning algorithms and/or one or more data collections. For example, the machine learning algorithms can perform perturbations to the initial design in silico to refine the design for the predetermined use of the diffractive decoder. In another example, data can be collected using the initial design of the diffractive decoder, and the data can be used, along with knowledge about the affects of changing the properties of the diffractive decoder, to iterate on the design of the diffractive decoder to better adapt the diffractive decoder for use in the predetermined application.

The design of the diffractive decoder may comprise use of an optical mind system (e.g., a system configured to aid in or perform the designing of a diffractive decoder). The optical mind system may comprise the one or more machine learning algorithms described elsewhere herein. For example, the optical mind system can be configured to utilize the one or more machine learning algorithms in the design of the diffractive decoder. In the example of FIG. 5 the optical mind system can intake parameters such as, for example, the present design of the diffractive decoder and the data produced by use of the diffractive decoder, to iterate on the design of the diffractive decoder, which new designs can be further iterated on in turn.

In this way, a diffractive decoder can be iteratively designed for a predetermined application, and the quality of the diffractive decoder for that application can be tuned and optimized.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method of detecting a biomolecule, comprising:
(i) providing a sample comprising said biomolecule;
(ii) directing electromagnetic radiation to said sample, thereby interacting said electromagnetic radiation with said biomolecule;
(iii) propagating said electromagnetic radiation through a diffractive decoder, thereby generating an optical output; and
(iv) detecting a presence or absence of said biomolecule in said sample based at least in part on said optical output.

2. The method of claim 1, wherein said diffractive decoder comprises a waveguide.

3. The method of claim 2, wherein said waveguide is a one dimensional or two dimensional waveguide.

4. The method of claim 1, wherein said diffractive decoder comprises a plurality of layers.

5. The method of claim 1, wherein said diffractive decoder comprises a plurality of nano-printed features.

6. The method of claim 1, wherein said biomolecule is selected from the group consisting of a nucleic acid molecule, a protein, and an antigen.

7. The method of claim 1, wherein said sample comprises one or more of blood, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, other excretions or body tissues, or any combination thereof.

8. The method of claim 1, wherein said detecting comprises use of an optical detector.

9. The method of claim 8, wherein said optical detector comprises a photomultiplier tube (PMT), photodiode, avalanche diode, single-photon avalanche diode, single-photon avalanche diode array, phototransistor, reverse-biased light emitting diode (LED), charge coupled device (CCDs), a time of flight (TOF) sensor, or complementary metal oxide semiconductor (CMOS) camera.

10. The method of claim 8, wherein said optical detector converts said optical output to an electrical output.

11. The method of claim 1, wherein said electromagnetic radiation comprises light.

12. The method of claim 11, wherein said light comprises multiple wavelengths of light.

13. The method of claim 11, wherein said light is structured light.

14. The method of claim 1, further comprising determining a quantity or concentration of said biomolecule.

15. The method of claim 1, wherein said electromagnetic radiation is transmitted through said sample.

16. The method of claim 1, wherein said electromagnetic radiation is reflected off said sample.

17. The method of claim 1, wherein (a)-(d) occurs in less than about 1 minute.

18. The method of claim 17, wherein (a)-(d) occurs in less than about 1 second.

19. The method of claim 18, wherein (a)-(d) occurs in less than about 0.1 seconds.

20. A diffractive decoder, comprising:
a waveguide comprising a plurality of physical features formed therein, wherein a physical arrangement of said plurality of physical features collectively defines a trained neural network, wherein said trained neural network is configured to propagate an input optical signal through said waveguide to generate an output optical signal.

21. The diffractive decoder of claim 20, further comprising a detector.

22. The diffractive decoder of claim 21, further comprising a plurality of detectors.

23. The diffractive decoder of claim 22, wherein said plurality of detectors are positioned at a plurality of locations in or adjacent to said waveguide.

24. The diffractive decoder of claim 21, further comprising a plurality of holes in said waveguide, wherein said plurality of holes are in optical communication with said detector.

25. The diffractive decoder of claim 21, wherein said detector comprises a photomultiplier tube (PMT), photodiode, avalanche diode, single-photon avalanche diode, single-photon avalanche diode array, phototransistor, reverse-biased light emitting diode (LED), charge coupled device (CCDs), or complementary metal oxide semiconductor (CMOS) camera.

26. The diffractive decoder of claim 20, wherein said waveguide is configured to propagate said input optical signal in one dimension.

27. The diffractive decoder of claim 20, wherein said waveguide is configured to propagate said input optical signal in two dimensions.

28. The diffractive decoder of claim 20, wherein said waveguide comprises gold, silver, a dielectric material, or any combination thereof.

29. The diffractive decoder of claim 20, wherein said waveguide is a cylinder.

30. The diffractive decoder of claim 20, wherein said waveguide is a prism.

31. The diffractive decoder of claim 20, wherein said waveguide is planar.

32. The diffractive decoder of claim 20, further comprising a mirror positioned within said waveguide.

33. The diffractive decoder of claim 32, wherein said mirror is configured to reflect said input optical signal back through said waveguide.

34. The diffractive decoder of claim 20, wherein said output optical signal is configured for use as illumination light.

35. The diffractive decoder of claim 20, wherein said output optical signal is configured to be detected by a detector.

36. The diffractive decoder of claim 20, wherein said physical features are not adjustable with respect to one another.

37. The diffractive decoder of claim 20, further comprising a sample area in optical communication with said waveguide.

38. The diffractive decoder of claim 37, wherein said sample area is in transmissive communication with said waveguide.

39. The diffractive decoder of claim 37, wherein said sample area is in reflective communication with said waveguide.

40. A system for detecting a biomolecule, comprising:
(v) an electromagnetic radiation source configured to direct electromagnetic radiation to a sample comprising said biomolecule, thereby interacting said electromagnetic radiation with said biomolecule;
(vi) a diffractive decoder configured to propagate said electromagnetic radiation, thereby generating an optical output;
(vii) a detector configured to detect said optical output; and
(viii) a computer processor operatively coupled to said detector, the computer processor programmed to detect a presence or absence of said biomolecule in said sample based at least in part on said detected optical output.

41. The system of claim 40, wherein said diffractive decoder comprises a waveguide.

42. The system of claim 41, wherein said waveguide is a one dimensional or two dimensional waveguide.

43. The system of claim 40, wherein said diffractive decoder comprises a plurality of layers.

44. The system of claim 40, wherein said diffractive decoder comprises a plurality of nano-printed features.

45. The system of claim 40, wherein said biomolecule is selected from the group consisting of a nucleic acid molecule, a protein, and an antigen.

46. The system of claim 40, wherein said sample comprises one or more of blood, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, other excretions or body tissues, or any combination thereof.

47. The system of claim 40, wherein said detecting comprises use of an optical detector.

48. The system of claim 47, wherein said optical detector comprises a photomultiplier tube (PMT), photodiode, avalanche diode, single-photon avalanche diode, single-photon avalanche diode array, phototransistor, reverse-biased light emitting diode (LED), charge coupled device (CCDs), or complementary metal oxide semiconductor (CMOS) camera.

49. The system of claim 47 or claim 48, wherein said optical detector converts said optical output to an electrical output.

50. The system of claim 40, wherein said electromagnetic radiation comprises light.

51. The system of claim 50, wherein said light comprises multiple wavelengths of light.

52. The system of claim 50, wherein said light is structured light.

53. The system of claim 40, further comprising determining a quantity or concentration of said biomolecule.

54. The system of claim 40, wherein said electromagnetic radiation is transmitted through said sample.

55. The system of claim 40, wherein said electromagnetic radiation is reflected off said sample.

56. The system of claim 40, wherein (a)-(d) occurs in less than about 1 minute.

57. The system of claim 56, wherein (a)-(d) occurs in less than about 1 second.

58. The system of claim 57, wherein (a)-(d) occurs in less than about 0.1 seconds.
